(19) United States
(12) Patent Application Publication
(10) Pub. No.: US 2022/0339119 A1
(43) Pub. Date: Oct. 27, 2022

(54) TRANSDERMAL MEDICAMENT

(71) Applicants: ELY NIGEL STRATEMEYER-TRINCZEK, Cowichan Bay (CA); JENELLE DIANE LECKY, Shawnigan Lake (CA)

(72) Inventors: ELY NIGEL STRATEMEYER-TRINCZEK, Cowichan Bay (CA); JENELLE DIANE LECKY, Shawnigan Lake (CA)

(21) Appl. No.: 17/601,358

(22) PCT Filed: Apr. 3, 2020
(86) PCT No.: PCT/CA2020/050452

§ 371 Date: Oct. 4, 2021

Related U.S. Application Data
(60) Provisional application No. 62/828,623, filed on Apr. 3, 2019.

(51) Int. Cl. A61K 9/70; A61K 31/05; A61K 47/20; A61K 47/10; A61K 47/12; A61K 47/14; A61K 47/26; B29C 65/48

(52) U.S. Cl. CPC A61K 9/7038; A61K 31/05; A61K 47/20; A61K 47/10; A61K 47/12; A61K 47/14; A61K 47/26; B29C 65/4825; B29K 2105/0035

(57) ABSTRACT

Transdermal medicaments containing cannabidiol (CBD) for use in the amelioration of conditions, including anxiety, depression, pain, inflammation, epilepsy, parkinson's disease, oxidative injury and nausea; and a method for preparing same.

### FIELD OF THE INVENTION

The present invention relates to the field of transdermal medicaments containing cannabidiol (CBD) for use in the amelioration of conditions, including anxiety, depression, pain, inflammation, epilepsy, Parkinson's disease, oxidative injury and nausea.

### BACKGROUND OF THE INVENTION

A transdermal patch is a medicated adhesive patch that is placed on the skin to deliver a dose of medication through the skin and into the bloodstream. A transdermal patch generally provides a controlled release of the medication into the patient, usually through either a porous membrane covering a reservoir of medication or through body heat melting thin layers of medication embedded in the adhesive. The main disadvantage to transdermal delivery systems relates to the fact that the skin is a very effective barrier; as a result, it is generally understood that only medications whose molecules are small enough to penetrate the skin can be administered using a transdermal patch.

The main types of transdermal patches include the Single-layer Drug-in-Adhesive type and the Multi-layer Drug-in-Adhesive type.

In the Single-layer Drug-in-Adhesive type, the adhesive layer also contains the drug. In this type of patch the adhesive layer not only serves to adhere the various layers together, along with the entire system to the skin, but is also responsible for the releasing of the drug. The adhesive layer is located between a temporary liner and a backing.

The multi-layer drug-in-adhesive patch generally has a layer akin to that in the single-layer system; but the multi-layer system multi-layer drug-in-adhesive patch includes at least one additional layer of drug-in-adhesive, with the layers usually separated by a membrane (but not in all cases). Generally, one of the layers is for immediate release of the drug and the other layer is for controlled release of the drug from the reservoir. As with the Single-layer Drug-in-Adhesive patch, the multi-layer drug-in-adhesive patch also has a temporary liner-layer and a permanent backing. The drug release from the multi-layer drug-in-adhesive patch depends on membrane permeability and diffusion of drug molecules.

### SUMMARY OF THE INVENTION

In one aspect, the present invention provides a transdermal patch which carries cannabidiol, as well as other ingredients, to the bloodstream. The patch is comprised of a backing, a non cross linking acrylic co-polymer pressure sensitive adhesive attached to one side of the backing, which contains natural anti inflammatory ingredients and a secondary layer which contains a carrier agent and cannabidiol. The cannabidiol is infused with the carrier agent on the secondary layer. The co-polymer and secondary layer diffuse into the bloodstream of the user. The transdermal patch may be used to treat anxiety, depression, pain, inflammation, epilepsy, oxidative injury and nausea.

In another aspect, the present invention provides a transdermal medicament including: a backing; an adhesive for adhering the patch to the user's skin; and cannabidiol (CBD) affixed to or intermingled with the adhesive and in an amount of 1-300 mg.

The transdermal medicament may include a carrier agent mixed with the cannabidiol. The carrier agent may be dimethyl sulphoxide.

One or more of the following may be intermingled with the adhesive: gingerol, shoagol, curcumin, carvacrol, ursolic acid, rosmarinic acid, baicalin and acetyl-11-keto-beta boswellic acid.

One or more of the following may be intermingled with the adhesive: 5-15 mg gingerol; 5-15 mg Shoagol; 5-15 mg curcumin; 5-15 mg carvacrol; 5-15 mg ursolic acid; 5-15 mg rosmarinic acid; 5-15 mg baicalin; and 5-15 mg acetyl-11-keto-beta boswellic acid.

One or more of the following may be intermingled with the adhesive: 10 mg gingerol; 10 mg Shoagol; 15 mg curcumin; 10 mg carvacrol; 5 mg ursolic acid; 5 mg rosmarinic acid; 5 mg baicalin; and 10 mg acetyl-11-keto-beta boswellic acid.

The cannabidiol (CBD) may be in an amount of 10, 20, 40, 60, 80 or 100 mg.

The transdermal medicament may include a carrier agent mixed with the cannabidiol, wherein the carrier agent is dimethyl sulphoxide; and wherein the mixed cannabidiol and dimethyl sulphoxide are affixed to the adhesive and comprise a distinct layer created by: mixing the dimethyl sulphoxide and the cannabidiol at a temperature of 40 to 50 degrees celsius; applying the mixed cannabidiol and dimethyl sulphoxide to the adhesive at a temperature of 25-30 degrees celsius; permitting the applied mixed cannabidiol and dimethyl sulphoxide, and the adhesive to thicken and adhere one to the other at a temperature of 20 degrees celsius or lower.

In another aspect, the present invention provides a method for making a transdermal medicament including: providing a backing; applying a co-polymer pressure sensitive adhesive to the backing; mixing dimethyl sulphoxide and cannabidiol at a temperature of 40 to 50 degrees celsius; applying the mixed cannabidiol and dimethyl sulphoxide to the adhesive at a temperature of 25 to 30 degrees celsius; permitting the applied mixed cannabidiol and dimethyl sulphoxide, and the adhesive to thicken and adhere one to the other at a temperature of no more than 20 degrees celsius.

The co-polymer pressure sensitive adhesive may contain one or more of: gingerol, shoagol, curcumin, carvacrol, ursolic acid, rosmarinic acid, baicalin and acetyl-11-keto-beta boswellic acid.

The method may include applying a release liner to the mixed cannabidiol and dimethyl sulphoxide, and the adhesive.

In another aspect, the present invention provides for use of a transdermal medicament comprising a backing, an adhesive for adhering the patch to a user's skin; and cannabidiol (CBD) in an amount of 1-300 mg and affixed to or intermingled with the adhesive, for amelioration of anxiety, depression, pain, inflammation, epilepsy, Parkinson's disease, oxidative injury and nausea.

In another aspect, the present invention provides for use of a transdermal medicament comprising a backing, an adhesive for adhering the patch to a user's skin; and cannabidiol (CBD) in an amount to provide a daily dose of 1-5 mg CBD, and affixed to or intermingled with the adhesive, for the promotion of general health and wellness.

In another aspect, the present invention provides for use of a transdermal medicament comprising a backing, an adhesive for adhering the patch to a user's skin; and cannabidiol (CBD) in an amount to provide a daily dose of 5 mg CBD per user kg, and affixed to or intermingled with the adhesive, for ameliorating the effects of withdrawal in addiction.

In another aspect, the present invention provides for use of a transdermal medicament comprising a backing, an adhesive for adhering the patch to a user's skin; and cannabidiol (CBD) in an amount to provide a daily dose of 0.8 to 5 mg CBD per user kg, and affixed to or intermingled with the adhesive, for ameliorating anxiety.

In another aspect, the present invention provides for use of a transdermal medicament comprising a backing, an adhesive for adhering the patch to a user's skin; and cannabidiol (CBD) in an amount to provide a daily dose of 5 mg CBD per user kg, and affixed to or intermingled with the adhesive, for ameliorating diabetes.

In another aspect, the present invention provides for use of a transdermal medicament comprising a backing, an adhesive for adhering the patch to a user's skin; and cannabidiol (CBD) in an amount to provide a daily dose of less than 160 mg CBD, and affixed to or intermingled with the adhesive, for promoting sleep onset.

In another aspect, the present invention provides for use of a transdermal medicament comprising a backing, an adhesive for adhering the patch to a user's skin; and cannabidiol (CBD) in an amount to provide a daily dose of more than 160 mg CBD, and affixed to or intermingled with the adhesive, for prolonging sleep.

In another aspect, the present invention provides for use of a transdermal medicament comprising a backing, an adhesive for adhering the patch to a user's skin; and cannabidiol (CBD) in an amount to provide a daily dose of 150-400 mg CBD, and affixed to or intermingled with the adhesive, for ameliorating psychotic symptoms associated with Parkinson's disease.

### DETAILED DESCRIPTION

Embodiments of the present invention include a two-layer transdermal patch having:

a) a backing (e.g., a 1.5 inch×1.5 inch Patch Export LLC FT-200 Polyethylene backing, White 5 mil 8″×10″ HDPE Liner Silicone Release 1 Side or 3M CoTran polyurethane monolayer film backing);
b) a non cross linking acrylic co-polymer pressure sensitive adhesive (preferably of moderate molecular weight) attached to one side of the backing, and containing gingerol, shoagol, curcumin, carvacrol, ursolic acid, rosmarinic acid, baicalin and acetyl-11-keto-beta boswellic acid;
c) a secondary layer overlying the co-polymer pressure sensitive adhesive and containing a carrier agent (dimethyl sulphoxide) and cannabidiol; and
d) a release liner (e.g., a Patch Export LLC 90 gm per square meter Semi-bleached densified kraft release liner).

Step 1: The co-polymer pressure sensitive adhesive is made and attached to the backing.

In a preferred embodiment, the co-polymer pressure sensitive adhesive contains per patch:

10 mg gingerol

### 10 mg Shoagol

15 mg curcumin
10 mg carvacrol
5 mg ursolic acid
5 mg rosmarinic acid
5 mg baicalin
10 mg acetyl-11-keto-beta boswellic acid

The co-polymer pressure sensitive adhesive may contain the following per patch:

5-15 mg gingerol

### 5-15 mg Shoagol

5-15 mg curcumin
5-15 mg carvacrol
5-15 mg ursolic acid
5-15 mg rosmarinic acid
5-15 mg baicalin
5-15 mg acetyl-11-keto-beta boswellic acid

Step 2: The secondary layer is made and placed to overlie the co-polymer pressure sensitive adhesive.

In a preferred embodiment, the secondary layer contains per patch: 16.6 mg dimethyl sulphoxide

20, 40, 60, 80 or 100 mg cannabidiol

The secondary layer may contain per patch:

5-30 mg dimethyl sulphoxide
10-300 mg cannabidiol

In preparing the secondary layer, the dimethyl sulphoxide is first mixed with the cannabidiol at a temperature of 40 to 50 degrees celsius. After mixing, the mixture is maintained at 25-30 degrees celsius as it is added to the patch to maintain it's viscosity and consistency. A suitable quantity of the mixture is placed on the co-polymer pressure sensitive adhesive and the release liner is applied. The finished product is then stored in a cool area, 20 degrees celsius or lower, to thicken and adhere properly to the backing.

In another embodiment, a single layer transdermal patch has:

a) a backing
b) a non cross linking acrylic co-polymer pressure sensitive adhesive combination containing gingerol, shoagol, curcumin, carvacrol, ursolic acid, rosmarinicacid, baicalin and acetyl-11-keto-beta boswellic acid, DMSO (dimethyl sulphoxide) and cannabidiol; and attached to one side of the backing.

The co-polymer pressure sensitive adhesive combination is made and attached to the backing essentially as indicated above with respect to the two layer transdermal patch. The combination preferably contains per patch;

10 mg gingerol

### 10 mg Shoagol

15 mg curcumin
10 mg carvacrol
5 mg ursolic acid
5 mg rosmarinic acid
5 mg baicalin
10 mg acetyl-11-keto-beta boswellic acid

### 16.6 mg Dimethyl Sulphoxide

20, 40, 60, 80 or 100 mg cannabidiol

Applicant believes that CBD is a multi-target medication, meaning it has diverse actions at a number of receptor sites that can interact with or counteract one another. These various receptor sites may be activated or inhibited at different doses, producing distinct profiles of effects. CBD appears to be triphasic, meaning CBD is believed to produce three distinct profiles of therapeutic effects at low, moderate, and high doses.

An example of distinct profiles of therapeutic effects with CBD is serotonin receptors—lower doses of CBD tend to enhance the function of serotonin receptors and thus improve anxiety, wheras high doses of CBD can inhibit those receptors and exacerbate anxiety. As well, what works for one individual or one condition may not work for another.

Although thinking of CBD as triphasic is a useful generalization, the picture in total is likely more complicated.

Applicant believes that the best practice is to start at the lowest dose possible and titrate upwards in no more than 10 mg increments—this will allow one to assess effects without missing their ideal dose. Increasing in small increments—10 mg—is understood to enable consumers to identify their “triphasic peak.”

General Health and Wellness: In the case of general health and wellness consumers, it's recommended to start very low, around 2.5-5 mg of CBD. Applicant believes that CBD can produce positive and healthful effects even at low doses, and possibly even microdoses (1-2.5 mg). If 5 mg is ineffective, doses should be titrated upwards in 10 mg increments.

Applicant believes microdosing of CBD increases sensitivity to endocannabinoids (e.g., AEA and 2-AG), essentially an effect akin to the opposite of tolerance.

Applicant believes that for ameliorating the effects of withdrawal in treatment for addiction, 300 mg CBD per day (for a 60 kg human) may be a suitable dosage, but confirmatory human testing is needed.

Applicant believes that for ameliorating anxiety 50-300 mg of CBD per day (for a 60 kg human), is likely a suitable dosage range. At doses below the therapeutic window CBD may be ineffective at reducing anxiety, and at doses above the therapeutic window anxiety may be exacerbated.

Applicant believes that for slowing or preventing the development of diabetes, 300 mg of CBD per day (for a 60 kg human, i.e., 5 mg/kg of body weight) may be a suitable dosage, but confirmatory human testing is needed.

Applicant believes that as a sleep aid, below 160 mg, CBD may help to promote sleep (reduce the time it takes to fall asleep), but doesn't seem to increase the quality or duration of sleep. Applicant believes that at and above 160 mg, CBD is able to induce and prolong sleep. Confirmatory human testing is needed. Applicant believes that doses should be titrated up by 10 mg increments every night until sleep comes easily.

Applicant believes that for ameliorating psychotic symptoms that may be associated with Parkinson's disease, 150-400 mg of CBD per day, is likely a suitable dosage range. Confirmatory human testing is needed.

The scope of the claims should not be limited by the preferred embodiments set forth in the examples, but should be given the broadest interpretation consistent with the description as a whole.

What is claimed is:

1. A transdermal medicament comprising:
a backing;
an adhesive for adhering the patch to the user's skin; and
cannabidiol (CBD) affixed to or intermingled with the adhesive and in an amount of 1-300 mg.

2. The transdermal medicament of claim 1, further comprising a carrier agent mixed with the cannabidiol.

3. The transdermal medicament of claim 2, wherein the carrier agent is dimethyl sulphoxide.

4. The transdermal medicament of claim 1, further comprising one or more of the following intermingled with the adhesive: gingerol, shoagol, curcumin, carvacrol, ursolic acid, rosmarinic acid, baicalin and acetyl-11-keto-beta boswellic acid.

5. The transdermal medicament of claim 1, further comprising one or more of the following intermingled with the adhesive: 5-15 mg gingerol; 5-15 mg Shoagol; 5-15 mg curcumin; 5-15 mg carvacrol; 5-15 mg ursolic acid; 5-15 mg rosmarinic acid; 5-15 mg baicalin; and 5-15 mg acetyl-11-keto-beta boswellic acid.

6. The transdermal medicament of claim 1, further comprising one or more of the following intermingled with the adhesive: 10 mg gingerol; 10 mg Shoagol; 15 mg curcumin; 10 mg carvacrol; 5 mg ursolic acid; 5 mg rosmarinic acid; 5 mg baicalin; and 10 mg acetyl-11-keto-beta boswellic acid.

7. The transdermal medicament of claim 1, wherein the cannabidiol (CBD) is in an amount of 10, 20, 40, 60, 80 or 100 mg.

8. The transdermal medicament of claim 1,
further comprising a carrier agent mixed with the cannabidiol, wherein the carrier agent is dimethyl sulphoxide; and
wherein the mixed cannabidiol and dimethyl sulphoxide are affixed to the adhesive and comprise a distinct layer created by:
mixing the dimethyl sulphoxide and the cannabidiol at a temperature of 40 to 50 degrees celsius;
applying the mixed cannabidiol and dimethyl sulphoxide to the adhesive at a temperature of 25-30 degrees celsius;
permitting the applied mixed cannabidiol and dimethyl sulphoxide, and the adhesive to thicken and adhere one to the other at a temperature of 20 degrees celsius or lower.

9. A method for making a transdermal medicament comprising:
providing a backing;
applying a co-polymer pressure sensitive adhesive to the backing;
mixing dimethyl sulphoxide and cannabidiol at a temperature of 40 to 50 degrees celsius;
applying the mixed cannabidiol and dimethyl sulphoxide to the adhesive at a temperature of 25 to 30 degrees celsius;
permitting the applied mixed cannabidiol and dimethyl sulphoxide, and the adhesive to thicken and adhere one to the other at a temperature of no more than 20 degrees celsius.

10. The method of claim 9, wherein the co-polymer pressure sensitive adhesive contains one or more of: gingerol, shoagol, curcumin, carvacrol, ursolic acid, rosmarinic acid, baicalin and acetyl-11-keto-beta boswellic acid.

11. The method of claim 9, further comprising applying a release liner to the mixed cannabidiol and dimethyl sulphoxide, and the adhesive.

12. Use of a transdermal medicament comprising a backing, an adhesive for adhering the patch to a user's skin; and cannabidiol (CBD) in an amount of 1-300 mg and affixed to or intermingled with the adhesive, for amelioration of anxiety, depression, pain, inflammation, epilepsy, Parkinson's disease, oxidative injury and nausea.

13. Use of a transdermal medicament comprising a backing, an adhesive for adhering the patch to a user's skin; and cannabidiol (CBD) in an amount to provide a daily dose of 1-5 mg CBD, and affixed to or intermingled with the adhesive, for the promotion of general health and wellness.

14. Use of a transdermal medicament comprising a backing, an adhesive for adhering the patch to a user's skin; and cannabidiol (CBD) in an amount to provide a daily dose of 5 mg CBD per user kg, and affixed to or intermingled with the adhesive, for ameliorating the effects of withdrawal in addiction.

15. Use of a transdermal medicament comprising a backing, an adhesive for adhering the patch to a user's skin; and cannabidiol (CBD) in an amount to provide a daily dose of 0.8 to 5 mg CBD per user kg, and affixed to or intermingled with the adhesive, for ameliorating anxiety.

16. Use of a transdermal medicament comprising a backing, an adhesive for adhering the patch to a user's skin; and cannabidiol (CBD) in an amount to provide a daily dose of 5 mg CBD per user kg, and affixed to or intermingled with the adhesive, for ameliorating diabetes.

17. Use of a transdermal medicament comprising a backing, an adhesive for adhering the patch to a user's skin; and cannabidiol (CBD) in an amount to provide a daily dose of less than 160 mg CBD, and affixed to or intermingled with the adhesive, for promoting sleep onset.

18. Use of a transdermal medicament comprising a backing, an adhesive for adhering the patch to a user's skin; and cannabidiol (CBD) in an amount to provide a daily dose of more than 160 mg CBD, and affixed to or intermingled with the adhesive, for prolonging sleep.

19. Use of a transdermal medicament comprising a backing, an adhesive for adhering the patch to a user's skin; and cannabidiol (CBD) in an amount to provide a daily dose of 150-400 mg CBD, and affixed to or intermingled with the adhesive, for ameliorating psychotic symptoms associated with Parkinson's disease.
